(19) United States
(12) Patent Application Publication
(10) Pub. No.: US 2020/0078492 A1
(43) Pub. Date: Mar. 12, 2020

(54) PROCESS FOR OBTAINING A FUNCTIONAL DERMAL SUBSTITUTE OF DECELLURIZED AMNIOTIC MEMBRANE FROM THE PLACENTA COMBINATION WITH KERATINOCYTES AND ITS USE AS AN AGENT FOR TISSUE REGENERATION OF THE SKIN

(71) Applicants: Eduardo ALVARO GALUE, Nuevo Leon (MX); Fernando Perez Chavez, Nuevo Leon (MX)

(72) Inventors: Eduardo ALVARO GALUE, Nuevo Leon (MX); Fernando Perez Chavez, Nuevo Leon (MX)

(21) Appl. No.: 16/243,289

(22) Filed: Jan. 9, 2019

(30) Foreign Application Priority Data
Sep. 7, 2018 (MX) MX/A/2018/010813

(51) Int. Cl. A61L 27/38; A61L 27/36

(52) U.S. Cl. CPC A61L 27/3839; A61L 27/3604; A61L 27/3641; A61L 2430/40; A61L 27/3813; A61L 27/3687; A61L 27/3691; A61L 27/3895

(57) ABSTRACT

This invention provides a process of obtaining a functional dermal substitute from decellularized amniotic membrane from placenta in combination with mammalian keratinocytes and its use as a tissue regeneration agent of skin, which the process for the generation of an artificial skin graft in the laboratory using specialized techniques in biotechnology such as the mammalian cells culture, preferably human, in which keratinocytes are cultured in aseptic conditions that form part of the epidermal layer of the skin, which are combined with a mesh rich in collagen which is obtained from cells that compose the structure of the skin in combination with the amniotic membrane of the placenta to generate two layers that can be used in burn patients or with skin lesions. Additionally, this technology considerably reduces the cost of treatment compared to the cost of current methods such as skin graft treatments (autografts or allograft) and the result of this process is a prompt cellular reorganization, remodeling, and re-epithelization of the wound resulting in the formation of new skin tissue and avoiding the formation of fibrotic tissue that results from contraction muscle.

### OBJECT OF THE INVENTION

The object of the present invention is a process for the generation of an artificial skin graft in the laboratory using biotechnology specialized techniques such as tissue cultivation, preferably human cells that are grown under aseptic conditions, the keratinocytes which are part of the epidermal layer of the skin, which are combined with a mesh of collagen (a structural protein of all tissues of the body) which is obtained from cells that make up the structure of the skin in combination with amniotic membrane obtained from placenta to generate two layers in the laboratory that can be used in burn patients or skin lesions.

The object of the invention lies in a method to generate a functional dermal substitute composed of decellularized amniotic membrane in combination with epidermal skin cells (keratinocytes) from neonatal foreskin (allogeneic cells) or tissue of the patient himself (autologous cells), or the method to generate a biological dressing from the same decellularization of the amniotic membrane without keratinocytes culture, which will function at first as a dermal substitute and the second as a cover or biological dressing for the regeneration and treatment of wounds of partial and/or total thickness caused by burns, diabetic ulcers or any skin lesions. This design is based on the technique of tissue bio-engineering of tissues created in the laboratory and its formulation is mainly based on a flexible type 1 collagen matrix obtained from the process of decellularization of the amniotic membrane obtained from placentas of mammal, preferably human of 36-38 weeks of gestation, pre-cultivated with human keratinocytes on its surface for the generation of a dermal substitute of skin for the re-epithelization and cicatrization of cutaneous lesions, that together will stimulate early regeneration and/or reconstruction of the skin tissue by forming a new extracellular matrix (dermis-epidermis). Additionally, this technology considerably reduces the cost of treatment compared to the cost of current methods such as skin graft treatments (autografts or allograft) and the result of this process is a prompt cellular reorganization, remodeling, and re-epithelization of the wound resulting in the formation of new skin tissue and avoiding the formation of fibrotic tissue result of muscle contraction.

### BACKGROUND

Currently, the use of allogeneic grafts and xenotransplants limits the regeneration of the skin due to inflammatory processes that originate from the immune rejection that involves the use of skin donors and the use of skin from animals. This causes the low availability of this biological material for the treatment of skin wounds and there is the disadvantage of susceptibility to infections. Within tissue engineering regeneration is usually initiated with the application of a biodegradable matrix or “biological scaffold” with a porous, trabecular or reticulate structure, which is placed in the damaged tissue to promote, in the micro-environment appropriately, the growth and propagation in situ of the surrounding healthy resident cells or of stem cells that can be implanted in that tissue or be incorporated into the biomaterial that integrates the “biological scaffold”, in order to accelerate the regeneration. In this combination, living cells supply the biological components, while the “scaffolding” material serves to support and favor cell proliferation (Dong, et al., 2004; Muschler, 2004); while stem cell therapy has given rise to a new type of treatment that can be categorized as regenerative cell therapies and is currently one of the most exciting topics in contemporary medicine and persists as an active trend within the research in regenerative medicine (Hernandez-Ramirez, 2009). With regard to the development and technologies of dermal substitutes is the U.S. Pat. No. 9,888,999B2, consisting of an allograft acellular and its process of preparation from dermis of newly deceased human, autograft or xenografts. The removal of cells is achieved by means of two detergents and is then treated with gamma rays. The CN102580153B patent refers to a method for producing allograft of acellular dermal matrices consisting of the processing of animal skins such as inactivation, degreasing, decellularization, packaging and irradiation for disinfection. In the subject of biological dressings, there is the patent of Chinese origin CN100408107C that is prepared from animal material including amniotic membrane, where the material is treated until removing the components that could cause rejection by the receiver. A patent application, US20130289724A1, describes the use of amniotic membrane and corium as a biological dressing, which includes at least one layer of amniotic membrane tissue or corium and a layer with adhesive that will be in contact directly with the wound. We also found a US20050107876A1 American patent application detailing a dermal substitute comprising biodegradable polymer such as collagen and biomaterial such as amniotic membrane, the method of preparation and use thereof. The dermal substitute for the invention can be applied to wounds that require skin grafting, for example, severe burns. The dermal substitute with amniotic membrane instead of a silicone membrane has several advantages, such as better bio compatibility, anti-inflammatory activity and promotion of wound healing activity and commercial use as a basal membrane. In addition, the structure can be used as a basic matrix for cell culture. There is also an international application with a WO2009156398A1 number detailing an ex vivo method to obtain a population of human keratinocytes derived from mammalian pluripotent stem cells, preferably human. This comprises a stage of co-cultivation of mammalian pluripotent stem cells, preferably human, with cells that favor ectodermal differentiation in the presence of an agent that stimulates epidermal induction and an agent that stimulates the terminal differentiation of the keratinocytes. This technological review leads to the conclusion that this application has novelty worldwide because none of the technologies found has the same characteristics. In addition, the proposed process has additional advantages to the above-mentioned patents.

In addition to this, we can find on the market other products based on the culture in vitro of human keratinocytes on lattices of bovine collagen type 1 previously seeded with human fibroblasts (APLIGRAF).

### BRIEF DESCRIPTION OF THE FIGURES

FIG. 1: shows a photograph of the method of separation of the amniotic membrane (Amnio) from human placenta. The amniotic membrane is a cover that encircles the placental tissue.

FIG. 2: shows a photograph of the amniotic membrane separated and isolated from the placenta.

FIG. 3: shows a photograph of human keratinocytes culture-neonatal (allogenic) on amniotic membrane on the surface in conditions of sterility and crop box decellularized.

FIG. 4: shows a photograph of the cultivation of human keratinocytes on decellularized amniotic membrane on the surface aseptically boxed crop with keratinocytes in the patient's own (autologous).

FIG. 5: shows a photo of the dermal substitute or amniotic membrane placed on Vaseline gauze for easy handling and transportation.

FIG. 6: shows a photograph of a histological cut of hematoxylin-eosin staining of dermal substitute.

### DETAILED DESCRIPTION OF THE INVENTION

The present invention relates to a method to generate a functional dermal substitute from the amniotic membrane of mammalian placenta decellularization biotechnology, preferably human, in combination with keratinocytes (allogeneic or autologous), and its use as a dermal substitute in the treatment and management of injuries, and in turn, use as a dressing or biological coverage based on the single use of the decellularized amniotic membrane without mammal keratinocytes, preferably human; both grafts used for regeneration and treatment of partial or full thickness wounds caused by burns, diabetic ulcers, or any skin lesions, which comprises the following stages:

a) Obtaining and Decellularization of an Amniotic Membrane from Mammalian Placenta, Preferably Human.

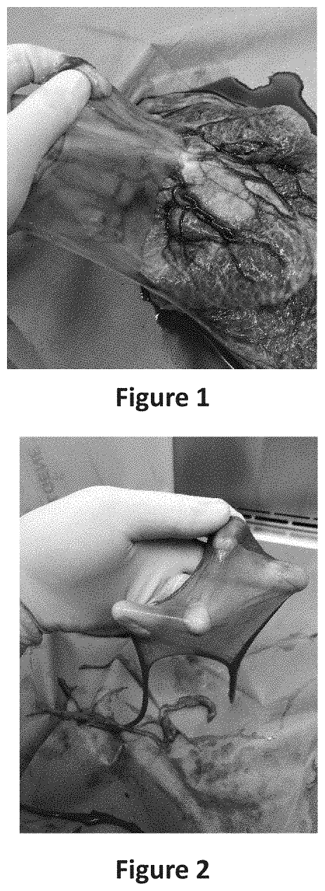

Gets an amniotic membrane (amnio) separated from the Corium, or embryo SAC of the placenta in mammal, preferably human, from 28-38 weeks of gestation, as shown in FIG. 1 and FIG. 2.

Afterwards, wash the amniotic membrane at least 3 times with a saline solution of phosphates (PBS) 1× containing 1-10% v/v solution of antibiotic/antimycotic preferably penicillin-streptomycin-gentamicin and incubated for at least 16 hours of 2-10 degrees Celsius.

Subsequently, after at least 16 hours, the amniotic membrane is mechanically sectioned or cut into 1 cm×1 cm pieces and up to 15×15 cm.

Incubate the amniotic membrane sections in an anionic surfactant component preferably in a sodium dodecyl sulfate solution (SDS) at a concentration of 0.03%-0.050% (w/v) dissolved in a phosphate buffered solution of 1× (w/v) and incubated from 20-27 hours at a temperature of 15 to 37 degrees Celsius.

Remove the sodium dodecyl sulfate solution (SDS) and wash at least 3 times with a phosphate buffer solution 1× (w/v).

Incubate, for at least 16 hours, the amniotic membrane sections in an enzymatic solution of DNAse from 0.025% to 0.050% (w/V) composed of buffered solution of phosphate buffer to 1× (w/v), magnesium chloride from 10 mM to 20 mM and pH 7 to 8.

Wash at least 3 times with a buffered phosphate buffer solution 1× (w/v) at 1% of an antibiotic-antifungal solution preferably penicillin-streptomycin-gentamicin.

Subsequently, to resuspend the amniotic membranes in a cryoprotectant composed of glycerol to a dissolution of 50 to 85% with a solution of cell culture medium preferably DMEM (Dubelcco's modified Eagle medium) high in glucose (4500 mg/L), 25 mm Hepes, 1% of an antibiotic-antifungal solution preferably penicillin-streptomycin-gentamicin and freeze the amniotic membranes at a temperature of −20 to −90 degrees centigrade.

Alternatively, the amniotic membranes are resuspended in a cryoprotectant composed of DMSO to a dissolution of 50% to 85% with a solution of cell culture medium preferably medium RPMI, Hank's solution, F-12, sodium chloride saline solution to 0.9%, or a buffered solution of phosphate buffered 1× all to 1% of an antibiotic-antifungal solution preferably penicillin-streptomycin-gentamicin and freeze the amniotic membranes at a temperature of −20 to −90 degrees centigrade.

Optionally it can incubate sections of amniotic membrane in a solution of sodium dodecyl sulfate (SDS) at a concentration of 0.03% to 0.050% (w/v) dissolved in a buffer of 1× phosphate buffered solution (w/v) and ethylenediaminetetraacetic acid (EDTA) at a concentration of 0.1% to 0.5% (w/v) and incubate 20 to 27 hours at a temperature of 15 to 37 degrees Celsius.

b) Obtain Allogeneic Keratinocytes from Neonatal Foreskin or Autologous Keratinocytes from a Mammal Sample, Preferably Human.

Obtain a sample of mammal skin, preferably human, in sterile conditions by means of a dermatome to obtain a sheet of skin, preferably of 0.015 inches or by means of a skin punch (punch) preferably of 2 mm to 8 mm, or to obtain a healthy neonatal foreskin by donation.

Subsequently, wash the tissue sample at least 3 times with a buffered phosphate buffer solution (PBS) at 1× containing 10% v/v of an antibiotic/antifungal solution preferably penicillin-streptomycin-gentamicin. Then, wash at least 3 times with a 1×PBS solution containing 1% v/V of antibiotic/antifungal. Preferably, the antibiotic/antifungal solution will be penicillin-streptomycin-gentamicin.

After washing the skin sample, incubate with an proteolytic enzyme of cellular disintegration, preferably dispasses, at a concentration of 1.79 to 5 units/mg and incubate 4° C. for at least 16 hours to obtain a layer of epidermis and a layer of dermis. After 16 hours, separate the two layers mechanically and place them in sterile and independent containers.

Separate and wash the epidermis layer in a separate container at least 3 times with the 1×PBS solution and incubate the epidermis layer in a trypsin solution at a concentration of 0.025% trypsin in a final volume of ethylenediaminetetraacetic acid (EDTA). For at least 5 minutes at a temperature of 37 QC to obtain a trypsinic epidermis.

Neutralize the trypsinic epidermis with 10%-50% of a human keratinocytes culture medium containing 10% fetal bovine serum and vigorously agitate for at least 30 seconds to obtain a cellular solution. Subsequently, filter the cellular solution by a nitrocellulose filter with a pore size of 40 μm to obtain the mammal keratinocytes, preferably human, suspended in the filtered product.

Optionally, mesenchymal stem cells can be used instead of keratinocytes, obtained from adipose tissue, peripheral blood, bone marrow, amniotic fluid, cotyledons of placenta, dental pulp, for the coating of the amniotic membrane for the generation of a functional dermal substitute.

c) In Vitro Proliferation of Keratinocytes in Suspension.

Culture the suspended keratinocytes in a cell culture vial coated with collagen type 1 with a cell culture medium for keratinocytes preferably a nutritious supplemented with bovine pituitary extract (BPE) 0.1-0.4% (v/v), mammalian epidermal growth factor, preferably human epidermal growth factor and recombinant (EGF) 0.125 to 0.2 ng/ml, mammalian insulin, preferably human insulin 1 to 5 mg/ml, hydrocortisone 0.1 to 0.33 mg/ml, mammalian transferrin, preferably human 1-10 mg/ml, epinephrine 0.1 A 0.39 mg/ml and calcium chloride (CaCl₂)) 0.15 to 0.2 mM.

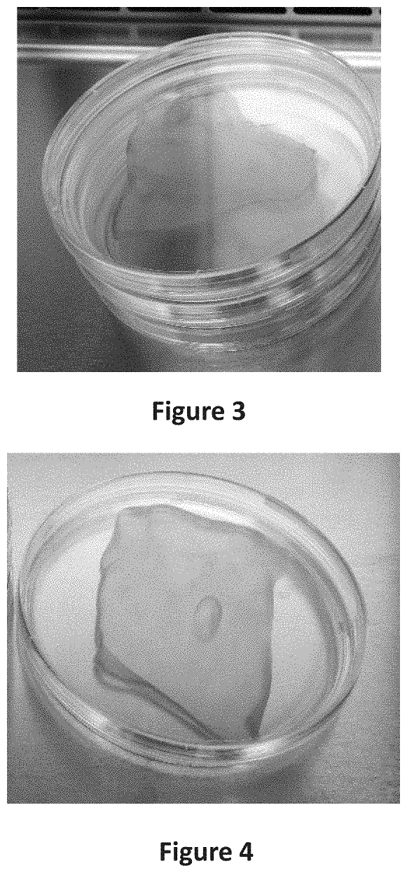

Incubate at 37° C., 95% relative humidity, 5% CO₂ and 20% O₂ for a period of at least 2 weeks to obtain an amount of 10-50×10⁶ of keratinocytes in monolayer, as shown in FIG. 3 and FIG. 4.

d) Functional Dermal Substitute Preparation.

Thaw the amniotic membrane at room temperature and wash for at least 3 times with a 1× phosphate buffer (PBS) solution containing 1% v/v of an antibiotic/antifungal solution preferably penicillin-streptomycin-gentamicin and place in a cell culture box.

Subsequently, wash with PBS 1× the container containing keratinocytess in monolayer for at least 2 times and incubate for at least 5 minutes with a solution of 0.025% to 0.05% of Trypsin in a final volume of ethylenediaminetetraacetic acid (EDTA) at a temperature of 37° C. Neutralize the Trypsinization solution by adding 50% of the volume of the container with special nutritive medium for keratinocytes supplemented with bovine pituitary extract (BPE) 0.1 to 0.4% (v/v), mammalian epidermal growth factor, preferably human and recombinant (EGF) 0.125 to 0.2 ng/ml mammalian insulin, preferably human, 1-5 mg/ml, hydrocortisone 0.1 to 0.33 mg/ml, transferrin of mammal, preferably human, 1-10 mg/ml, epinephrine 0.1 to 0.39 mg/ml and calcium chloride (CaCl₂)) 0.15-0.2 mM, 10-20% v/V autologous serum or fetal bovine serum pharmaceutical grade to obtain keratinocytes in suspension. Place 100% of the volume of the keratinocytes in a container and centrifuge at 1.500 revolutions per minute after from 5 to 10 minutes to obtain a keratinocytes cell pellet.

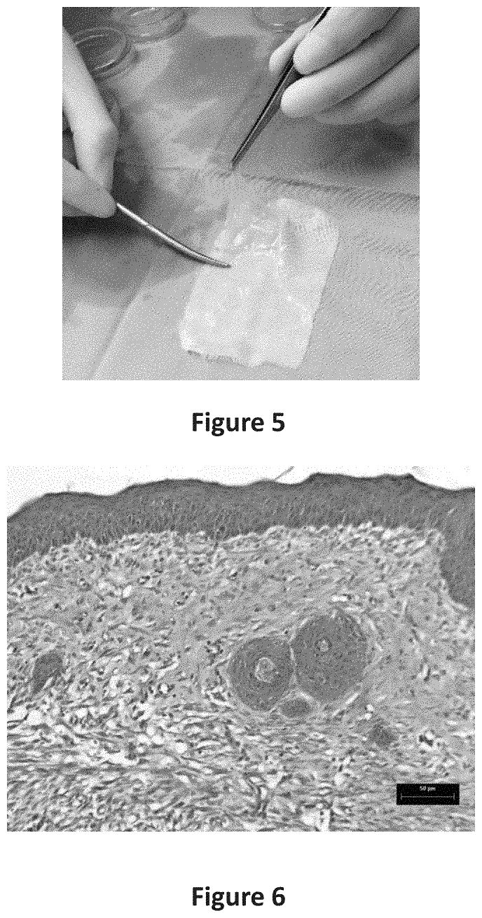

Resuspend the keratinocytes cell pellet in 1 to 10 ml of special cell culture medium for keratinocytes, count in Neubauer chamber at a 1:1 dilution with methylene blue and sow the keratinocytes in suspension on the decellularized amniotic membrane with a density of 5,000-50,000,000 keratinocytes per square centimeter and incubate at 37° C., 95% relative humidity, 5% CO₂ and 20% O₂ for a period of 1 to 2 weeks for the generation of a functional dermal replacement with keratin stratification in the surface of the amniotic membrane. This incubation of the amniotic membrane with the keratinocytes is carried out in a flask, petri dish or sterile container intended for the cultivation of static tissues or by the process of air-liquid interface (Liquid air interface) for the generation of the stratification of the epidermis on the surface of the decellularized amniotic membrane, to obtain the functional dermal substitute, as shown in FIG. 5 and FIG. 6.

Optionally, prior to the planting of keratinocytes in suspension, the decellularized amniotic membrane is coated with a collagen type 1 solution in sterile grade water at a concentration of 0.01% to 0.1% and incubated at room temperature or 37 degrees centigrade prior to the planting of the keratinocytes.

Also, the decellularized amniotic membrane can be coated or cultivated with stem cells from the periphery of the mammal cornea, preferably human.

### Example 1. Preferential Mode of Invention

The functional dermal substitute is composed of a matrix rich in collagen type 1 that is obtained from the decellularization of amniotic membrane of human placenta coated or cultivated on its surface with human keratinocytes allogeneic or autologous to generate an epidermal stratification prior to its cutaneous implantation. This dermal substitute is composed of a flexible collagen mesh from decellularization with anionic surfactant components that will eliminate all the structures, tissues and cell lines of the amniotic membrane to cause immune rejection. And it can be used as a donation, and in particular will present a synthesis of keratin on its surface with cutaneous stratification promoted by the keratinocytes cultured on its surface which will be cultivated for at least 1 month to generate the epidermis on the collagen matrix. This culture of functional dermal substitute can be in static conditions within a culture jar or petri dish or by the known liquid-air interface (Liquid air interface) method from a Transwell type culture vial in an incubator that provides the conditions of 90% moisture, 5% CO₂ to 37 degree Centigrade.

### Example 2. Functional Dermal Substitute Manipulation Mode

In aseptic conditions, the cell culture vial is opened horizontally, the cell culture medium is aspirated and a Vaseline gauze is placed on the surface of the dermal substitute for easy manipulation. Subsequently, the use of tweezers is gently peeled through the 4 corners or from the obtaining of the dermal substitute. It was possible to transport in sterile and sealed petri dishes to the operating room to be implanted in patients with cutaneous lesions.

In turn, the dermal substitute can be kept frozen for more than 1 year in a range of 70° C. to 100° C., once mounted on a Vaseline gauze inside a Tyvek bag for medical device and impregnated with a cryo-protector solution to be kept in ultra congelador for a period of time of at least one year. From the use of a frozen dermal substitute, it is thawed at room temperature, and in aseptic conditions in the operating room, the Tyvek bag is carefully opened and saline solution is placed or the dermal substitute is washed with sodium chloride saline at 0.9% (Physiological solution), to remove the Cryo protector and then place the skin substitute on the lesion to be treated.

In turn, the decellularized amniotic membrane can be used as a biological dressing for the coverage of open wounds.

### Example 3. Preferential Use of the Invention

This functional dermal substitute is based on the well-orchestrated combination of a mesh or collagen matrix that is obtained from the decellularization of the amniotic membrane of human placenta with the planting or growth of human keratinocytes over its surface for the generation of a dermal substitute, which make up the skin's native and function by the production of collagen fibers type I and IV (fibroblasts), production of Keratin (keratinocytes). All this, in combination with the use of the decellularized amniotic membrane, that will provide flexibility, support and manageability to the dermal substitute (two-dimensional) to be implanted and which will generate the vascularization at the time of being implanted, because it has growth factors that stimulate the production of new extracellular matrix in the wounds treated in patients with cutaneous pathologies decreasing the time and quality of re-epithelization of the skin. The dermal substitute comes to replace the use of alo, xeno and autografts as many times as the obtaining of skin from the same patient or the use of skin banks that is limited or not available without taking into account the use of skin of other species (bovine origin).

The preferential use of this innovation is based on the implantation of dermal substitute in cutaneous lesions with the potential to create new extracellular matrix cutaneous along with its functionality of the use of decellularized amniotic membrane which is a mesh rich in collagen that will promote the production of cutaneous dermis.

Its mechanism of action is based on a skin lesion caused by the removal of cancerous tissue, skin burns of partial or total thickness, venous diabetic ulcers, removal of skin scars, etc.

In turn, the decellularized amniotic membrane has the potential to use as a biological dressing on wounds or lesions of the skin at the time of not being cultivated with human keratinocytes, in order to function as a cover and the wound is not exposed to infections.

Having described enough my invention, I consider it as a novelty and therefore claim as my exclusive property, the content in the following claims:

1. A process for obtaining a functional dermal substitute from a decellularized amniotic membrane from a placenta in combination with keratinocytes, the method comprising the steps of:
a) obtaining and decellularization of the amniotic membrane from a mammal placenta;
b) obtaining allogeneic keratinocytes from a neonatal foreskin or an autologous keratinocyte from a patient sample;
c) in vitro proliferation of keratinocytes in suspension; and
d) preparation of functional dermal substitute.

2. The process according to claim 1, wherein in step a) the amniotic membrane is obtained separating from the corium or embryo sac of the mammal placenta and washing the amniotic membrane at least 3 times with a saline solution of phosphates (PBS) 1× containing 1-10% v/v of penicillin-streptomycin-gentamicin, incubated for at least 16 hours of 2-10 degrees celsius and mechanically sectioned in pieces of 1 cm×1 cm and up to 15×15 cm.

3. The process according to claim 1, wherein in the step a) the sections of the amniotic membranes are decellularizing by an incubation process of sodium dodecyl sulfate (SDS) solution at a concentration of 0.03% 0.050% (w/v) dissolved in a solution buffered 1× phosphate buffer (w/v) incubating for 20 to 27 hours at a temperature of 15 to 37 degrees centigrade, then washing 3 times with a solution of buffered 1× phosphate buffer (w/v), incubating for at least 16 hours in an enzymatic solution of DNase from 0.025% to 0050% (w/v) composed of buffered phosphate buffer solution 1× (w/V), 10 mm-20 mm Magnesium chloride, pH 7 to 8, and finally by washing at least 3 times with a buffered phosphate buffer solution 1× (wvV) 1% of an antibiotic-antifungal preferably penicillin-streptomycin solution-gentamicin for cryopreserve them at a temperature of −20 to −90 degrees celsius from a cryoprotectant composed of a glycerol solution of 50% to 85% with cell culture DMEM medium preferably (Dubelcco complete modified Eagle medium) high in glucose (4500 mg/L), 25 MM Hepes, 1% of a solution of antibiotic-antifungal preferably penicillin-streptomycin-gentamicin.

4. The process according to claim 3, wherein the sections of amniotic membrane are optionally incubated in a sodium dodecyl sulfate solution (SDS) at a concentration of 0.03%-0.050% (w/V) dissolved in a buffered solution of 1× (w/v) and ethylenediaminetetraacetic acid (EDTA) phosphates at a concentration of 0.1%-0.5% (w/v) to be incubated for a period of 20 to 27 hours at a temperature of 15 to 37 degrees Celsius.

5. The process according to claim 4, wherein the process obtains a decellularized membrane of a human, free of any cell line obtaining a mesh of collagen.

6. The process according to claim 3, wherein optionally the cryoprotectant includes DMSO, RPMI, Hank's solution, F-12, sodium chloride saline solution at 0.9%, or a buffered solution of phosphate buffered 1× all to 1% of penicillin-streptomycin-gentamicin.

7. The process according to claim 1, wherein in step b) human keratinocytes are isolated from autologous tissue from a sample of neonatal foreskin.

8. The process according to claim 1, wherein in step b), the tissue sample is washed at least 3 times with a 1× phosphate buffered (PBS) solution containing 10% v/v of an antibiotic/antifungal solution preferably; then washing at least 3 times with a 1×PBS solution containing 1% v/v of antibiotic/antifungal.

9. The process according to claim 8, wherein the solution antibiotic-antifungal is a penicillin-streptomycin-gentamicin solution.

10. The process according to claim 1, wherein in step b) is incubated with dispasses, for at least 16 hours to obtain a layer of epidermis and a layer of separating the two layers mechanically and placing in sterile and independent containers.

11. The process according to claim 1, wherein in step c) are cultured the keratinocytes in suspension in a cell culture vial coated with collagen type 1 with a cell culture medium for keratinocytes and incubated at 37° C., 95% relative humidity, 5% CO₂ and 20% O₂ for a period to obtain a quantity of 10 to 50×10⁶ of keratinocytes in monolayer.

12. The process according to claim 11, wherein the cell culture medium is supplemented with bovine pituitary extract (BPE) 0.1-0.4% (v/v), mammalian epidermal growth factor, and recombinant (EGF) 0.125 to 0.2 ng/ml mammalian insulin, preferably human, 1-5 mg/ml, hydrocortisone 0.1-0.33 mg/ml, mammal transferrin including human 1-10 mg/ml epinephrine 0.1-0.39 mg/ml and calcium chloride (CaCl₂)) 0.15-0.2 mM; incubate at 37° C., 95% relative humidity, 5% CO₂ and 20% O₂ for a period of to obtain a quantity of 10-50×10⁶ of keratinocytes in monolayer.

13. The process according to claim 12, wherein the process obtains a pure line of keratinocytes in vitro.

14. The, according to claim 1, wherein in step d) the functional dermal substitute is prepared by washing the amniotic membrane decellularized at least 3 times with a buffered phosphate buffer solution (PBS) at 1× containing 1% v/v of an antibiotic/antifungal solution preferably penicillin-streptomycin-gentamicin and placing in a cell culture box.

15. The process according to claim 1, wherein in step d) the dermal substitute function is prepared by culturing the keratinocytes cell pellet on the decellularized amniotic membrane with or without collagen coating type 1 with a minimum density of 5.000 keratinocytes per centimeter squared in a jar, petri dish or sterile container intended for the static cultivation of tissues or by cultivation from the process of air-liquid interface (Liquid air interface) to 37° C., 95% relative humidity, 5% CO₂ and 20% O₂ for a period of 1 to 2 weeks for the generation of the stratification of the epidermis on the surface of the decellularized amniotic membrane.

16. The process according to claim 15, wherein prior to sowing the keratinocytes, the decellularized amniotic membrane is coated with a type 1 collagen solution.

17. The process according to claim 15, wherein the nutritive medium for the generation of dermal substitute is supplemented with bovine pituitary extract (BPE) 0.1-0.4% (v/v), human mammalian epidermal growth factor, and recombinant (EGF) 0.125-0.2 ng/ml human mammalian insulin 1-5 mg/ml, hydrocortisone 0.1-0.33 mg/ml, human transferrin 1-10 mg/ml epinephrine from 0.1 to 0.39 mg/ml and calcium chloride (CaCl₂)) from 0.15 to 0.2 mM.

18. The process according to claim 15, wherein the incubation of the decellularized amniotic membrane with keratinocytes is carried out in a petri dish, container, or sterile vial destined to culture of static tissue or by the process of air-liquid interface for the generation of the stratification of the epidermis on the surface of the decellularized amniotic membrane.

19. A method for reepithelization of skin injuries comprising administering to a skin a functional dermal substitute from decellularized amniotic membrane from placenta in combination with keratinocytes obtained through the process included in claim 1.

20. The process according to claim 1, wherein the decellularized amniotic membrane alone can be used as a biological cover, skin dressing, or wound dressing on skin lesions.
